(19) United States
(12) Patent Application Publication
(10) Pub. No.: US 2022/0218270 A1
(43) Pub. Date: Jul. 14, 2022

(54) SYSTEMS, METHODS AND COMPUTER PROGRAM PRODUCTS FOR SKIN LIQUID LEVELS ESTIMATION

(71) Applicant: TriEye Ltd., Tel Aviv (IL)

(72) Inventors: Elior Dekel, Tel Aviv (IL); Roni Dobrinsky, Tel Aviv (IL)

(21) Appl. No.: 17/464,715

(22) Filed: Sep. 2, 2021

Related U.S. Application Data
(60) Provisional application No. 63/072,260, filed on Aug. 31, 2020.

(51) Int. Cl. A61B 5/00; G01N 21/3554; G06T 7/00; G06T 7/50; G06T 7/70; G06K 9/00; H04N 5/225

(52) U.S. Cl. CPC A61B 5/443; G06T 2207/10048; G06T 7/0012; G06T 7/50; G06T 7/70; G06K 9/00221; H04N 5/2256; A61B 5/0075; A61B 5/4875; A61B 5/0077; G01N 2201/063; G06T 2207/30088; G06T 2207/30201; G06T 2207/10152; G01N 21/3554

(57) ABSTRACT

Method and system for estimating skin liquid levels, comprising acquiring a plurality of spectrally distinct images of a skin of a body part, and for each area out of a plurality of areas on the body part: obtaining, from the plurality of spectrally distinct images, corresponding spectrally distinct light levels of the area, acquiring an angular orientation of the area, and based on the spectrally distinct light levels and the angular orientation of the area, determining a skin liquid level for the area, and, based on the skin liquid level for each area, generating a skin liquid levels map for the body part indicative of the skid liquid levels of the plurality of areas on the body part.

### CROSS REFRENCE TO RELATED APPLICATIONS

This application claims priority from U.S. provisional patent application Ser. No. 63/072,260 filed Aug. 31, 2020, which is incorporated herein by reference in its entirety.

### FIELD

This disclosure related to systems, methods and computer program products for skin liquid levels estimation, and especially to systems, methods and computer program products for contactless skin liquid levels estimation based on spectrally distinct images.

### SUMMARY

In various embodiments, there is a provided a method for estimating skin liquid levels, the method comprising acquiring a plurality of spectrally distinct images of a skin of a body part; and for each area out of a plurality of areas on the body part: obtaining, from the plurality of spectrally distinct images, corresponding spectrally distinct light levels of the area, acquiring an angular orientation of the area, and based on the spectrally distinct light levels and the angular orientation of the area, determining a skin liquid level for the area, and, based on the skin liquid level for each area, generating a skin liquid levels map for the body part indicative of the skid liquid levels of the plurality of areas on the body part.

In some embodiments, the skin liquid levels of the plurality of areas are indicative of hydration levels of the plurality of areas.

In some embodiments, the skin liquid levels of the plurality of areas are indicative of sebum levels of the plurality of areas.

In some embodiments, the acquiring of the angular orientation of the area comprises processing depth data captured by a detector that captured at least one of the spectrally distinct images.

In some embodiments, the acquiring of the angular orientations for the plurality of areas comprises applying a face-recognition algorithm to the body part, and assigning different angular orientations to different areas based on the results of the face recognition algorithm.

In some embodiments, the acquiring of the angular orientations for the plurality of areas comprises retrieving previously sampled 3D information of the body part from a memory storage, and mapping the 3D information to at least one of the spectrally distinct images.

In some embodiments, the same spectrally distinct light levels are obtained for a first area and a for a second area of the body part, wherein different angular orientations are acquired for the first area and for the second area, and wherein the method comprises determining a first skin liquid level for the first area and a second skin liquid level for the second area, the first skin liquid level and the second skin liquid level differing by at least 5%.

In some embodiments, the method further comprises illuminating the body part with spectrally distinct light beams, and capturing the spectrally distinct images corresponding to the spectrally distinct light beams.

In some embodiments, the method further comprises capturing the spectrally distinct images using a detector array having a plurality of spectrally distinct filters.

In some embodiments, at least one of the spectrally distinct images depicts a reflection target, wherein the generating of the skin liquid levels is further based on reflection levels of the reflection targets in the at least one of the spectrally distinct images.

In some embodiments, the method is a computer-implemented method for estimating skin liquid levels, comprising executing on a processor the steps of the method.

In some embodiments, at least two of the spectrally distinct images are indicative of detected light in infrared parts of the spectrum between 1000-1500 nm.

In various embodiments, there is provided a system comprising a processor configured to perform any of the methods as above or below.

In some embodiments, the system further comprises a 3D processing module operable to process depth data captured by a detector which captured at least one of the spectrally distinct images, for determining angular orientations of different body part areas.

In some embodiments, the processor is operable to execute a face-recognition algorithm to the body part, and to assigning different angular orientations to different areas based on the results of the face recognition algorithm.

In some embodiments, for the same spectrally distinct light levels are obtained for a first area and a for a second area of the body part, different angular orientations are acquired for the first area and for the second area, and wherein the processor determines a first skin liquid level for the first area and a second skin liquid level for the second area, the first skin liquid level and the second skin liquid level differing by at least 5%.

In some embodiments, the system further comprises at least one detector for capturing the spectrally distinct images corresponding to the spectrally distinct light beams.

In some embodiments, the system further comprises at least one light source for illuminating the body part with spectrally distinct light beams, and at least one detector for capturing the spectrally distinct images corresponding to the spectrally distinct light beams.

In some embodiments, at least one of the spectrally distinct images depicts a reflection target, wherein the processor is configured to generate the skin liquid levels further based on reflection levels of the reflection target in the at least one of the spectrally distinct images.

In some embodiments, in a system, at least two of the spectrally distinct images are indicative of detected light in infrared parts of the spectrum between 1000-1500 nm.

In some embodiments, the system is a portable communication device which comprises the processor and at least one detector for capturing the spectrally distinct images corresponding to the spectrally distinct light beams.

In some embodiments there is provided a program storage device readable by machine, tangibly embodying a program of instructions executable by the machine to perform a method for estimating skin liquid levels as above or below.

### BRIEF DESCRIPTION OF THE DRAWINGS

Non-limiting examples of embodiments disclosed herein are described below with reference to figures attached hereto that are listed following this paragraph. Identical structures, elements or parts that appear in more than one figure may be labeled with a same numeral in all the figures in which they appear. The drawings and descriptions are meant to illuminate and clarify embodiments disclosed herein, and should not be considered limiting in any way. All the drawings show devices or flow charts in accordance with examples of the presently disclosed subject matter. In the drawings:

FIG. 1 illustrates an embodiment of a system for estimating skin liquid levels, in accordance with examples of the presently disclosed subject matter;

FIG. 2 illustrates the system of FIG. 1 and its operation when determining skin liquid levels in a body part, in accordance with examples of the presently disclosed subject matter;

FIG. 3 illustrates an embodiment of a method for estimating skin liquid levels, in accordance with examples of the presently disclosed subject matter.

### DETAILED DESCRIPTION

In the following detailed description, numerous specific details are set forth in order to provide a thorough understanding of the disclosure. However, it will be understood by those skilled in the art that the present disclosure may be practiced without these specific details. In other instances, well-known methods, procedures, and components have not been described in detail so as not to obscure the present disclosure.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “processing”, “calculating”, “computing”, “determining”, “generating”, “setting”, “configuring”, “selecting”, “defining”, or the like, include action and/or processes of a computer that manipulate and/or transform data into other data, said data represented as physical quantities, e.g. such as electronic quantities, and/or said data representing the physical objects.

The terms “computer”, “processor”, and “controller” should be expansively construed to cover any kind of electronic device with data processing capabilities, including, by way of non-limiting example, a personal computer, a server, a computing system, a communication device, a processor (e.g. digital signal processor (DSP), a microcontroller, a field programmable gate array (FPGA), an application specific integrated circuit (ASIC), etc.), any other electronic computing device, and or any combination thereof.

The operations in accordance with the teachings herein may be performed by a computer specially constructed for the desired purposes or by a general-purpose computer specially configured for the desired purpose by a computer program stored in a computer readable storage medium.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

In embodiments of the presently disclosed subject matter one or more stages illustrated in the figures may be executed in a different order and/or one or more groups of stages may be executed simultaneously and vice versa. The figures illustrate a general schematic of the system architecture in accordance with an embodiment of the presently disclosed subject matter. Each module in the figures can be made up of any combination of software, hardware and/or firmware that performs the functions as defined and explained herein. The modules in the figures may be centralized in one location or dispersed over more than one location.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Disclosed below are systems and methods for determining skin liquid levels (e.g., hydration levels, sebum levels), which enable determining of such levels using images acquired by a contactless optical inspection system (such as a camera, photodetector array (PDA), and so on). The systems and methods are based on spectrally distinct images of a body part of a subject (e.g., a person, an animal) Spectrally distinct images are images that include information of light of different parts of the electromagnetic spectrum. A spectrally distinct image may include information of detected light within a spectral range (e.g. λⱼ-λₖ) which may be relatively narrow (e.g., having range of a few nanometers) or wider (e.g., tens or hundreds of nanometers). While not necessarily so, the spectral ranges of the spectrally distinct images may be non-overlapping.

For example, a first spectrally distinct image may detect light from about 1200±4 nm, a second spectrally distinct image may detect light from about 2080±4 nm, and a third may detect light from about 2100±4 nm. In another example a first spectrally distinct image may detect light from about 1200-1350 nm, a second spectrally distinct image may detect light from about 2080-1360 nm, and a third may detect light from about 2100-1420 nm. In yet another example a first spectrally distinct image may detect light from about 1200-2080 nm, a second spectrally distinct image may detect light from about 1280-2100 nm, and a third may detect light from about 1700±6 nm. Clearly, those are just three examples and are not limiting in any way. Optionally, at least two of the spectrally distinct images are indicative of detected light in infrared parts of the spectrum between 1000-1500 nm.

It is noted that in some implementations, one or more of the spectrally distinct images may include information of detected light within two or more distinct spectral ranges (e.g., 2100 nm-1420 nm and also 1500 nm-1520 nm). Such a compound range is also spectrally distinct from the spectral ranges (single or compound) of at least one other image out of the plurality of spectrally distinct images used in the detection. It is noted that some images used in the detection may represent light in similar spectral range, as long as sufficient number of images are spectrally distinct from one another. It is noted that in at least some of the implementation, one or more of following conditions are met:

a. The first spectrally distinct image includes information from a first spectral range that is not represented in the second spectrally distinct image, and the second spectrally distinct image includes information from a second spectral range that is not represented in the first spectrally distinct image.

b. The first spectrally distinct image includes information from a first spectral range that is not represented in the second spectrally distinct image nor in the third image; the second spectrally distinct image includes information from a second spectral range that is not represented in the first spectrally distinct image nor in the third spectrally distinct image; and the third spectrally distinct image includes information from a third spectral range that is not represented in the first spectrally distinct image nor in the second spectrally distinct image.

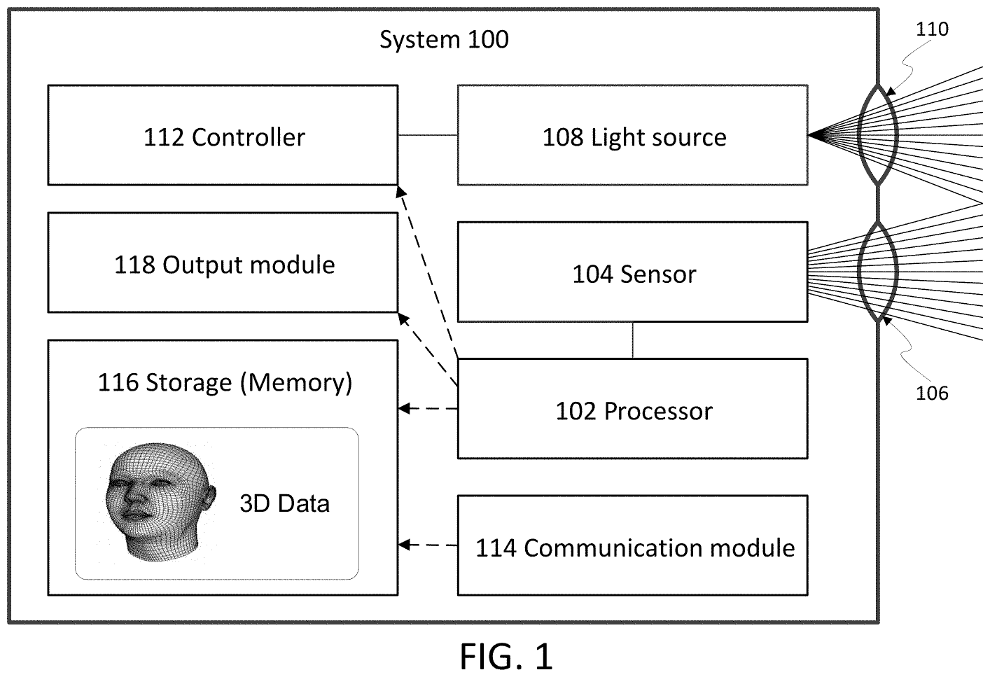

FIG. 1 illustrates system 100 for estimating skin liquid levels, in accordance with examples of the presently disclosed subject matter. System 100 includes at least a processor 102, and may include various additional component such as (but not limited to) any combination of one or more of the following optional components:

c. At least one sensor 104 operable to detect one or more spectrally distinct images which are processed by the processor for determining skin liquid levels of a body part. A single sensor 104 may acquire image data in one or more spectrally distinct spectral ranges. Examples of sensors 104 include cameras, focal plane arrays (FPAs), and so on.

d. Inbound optics 106 for directing light from a field of view (FOV) of system 100 towards sensor 104 and/or for manipulating the incoming light prior to impinging of the light on sensor 104. Inbound optics 106 may include any suitable type of optical components such as mirrors, lenses, prisms, optic fibers, spectral filters, polarizers, other filters, windows, retarders, and so on.

e. At least one light source 108, for illuminating objects in the FOV of system 100. Different types of light sources may be used, such as laser, light emitting diode (LED), and so on. If light source 108 is implemented, it can emit light in one or more of the spectrally distinct spectral ranges detected by the one or more sensors 104. Optionally, light source 108 may also emit light which is not detectable by sensor 104, or which is filtered out prior to reaching sensor 104.

f. Outbound optics 110 for directing light from a light source 108 towards the field of view of system 100 and/or for manipulating the emitted light prior to being emitted from system 100. Outbound optics 110 may include any suitable type of optical components such as mirrors, lenses, prisms, optic fibers, spectral filters, polarizers, other filters, windows, retarders, and so on.

g. Controller 112 for controlling operations of various components of system 100 (e.g., light source 108, sensors 104, communication module 114), and possibly also of external system (e.g., synchronizing operation of an external light source if implemented, synchronizing operations of an external sensors if implemented, and so on). Controller 112 may be implemented by any suitable combination of one or more of hardware, software, and/or firmware, and may include digital components, analogue components, or a combination thereof. For example, controller 112 may be a computer, a PCB, a software-on-chip (SOC) module, and so on.

h. Communication module 114, which may be used for inbound communication (e.g., receiving information from an external sensor, external controller, and so on), for outbound communication (e.g., for controlling external systems, for providing computation outputs, and so on). Any suitable standard of communication may be implemented, such as Bluetooth, Wi-Fi, LAN, WAN, and so on. Communication module 114 may implement wired communication, wireless communication, or both.

i. Memory module 116, for storing and retrieving of data. Examples of data which may be stored are the spectrally distinct images data, processing outputs, hydration levels, and so on. Any suitable memory module may be used, such as volatile memory, non-volatile memory, flash memory magnetic tape, and so on.

j. Output module 118 for outputting data to a user or anther system, such as hydration levels, system state, detected images, and so on. Output module 118 may include a display (monitor), speaker, or any other suitable form of output (e.g., indicator LED lights).

k. Any other component, many of which are custom in the art, such as power source, casing, user interface, and so on. Many such components are known in the art, and are not included for reasons of simplicity and clarity.

While system 100 may be a dedicated system, it may optionally be implemented in a system having a wider range of capabilities (e.g., a camera, computer, smartphone, car, and so on).

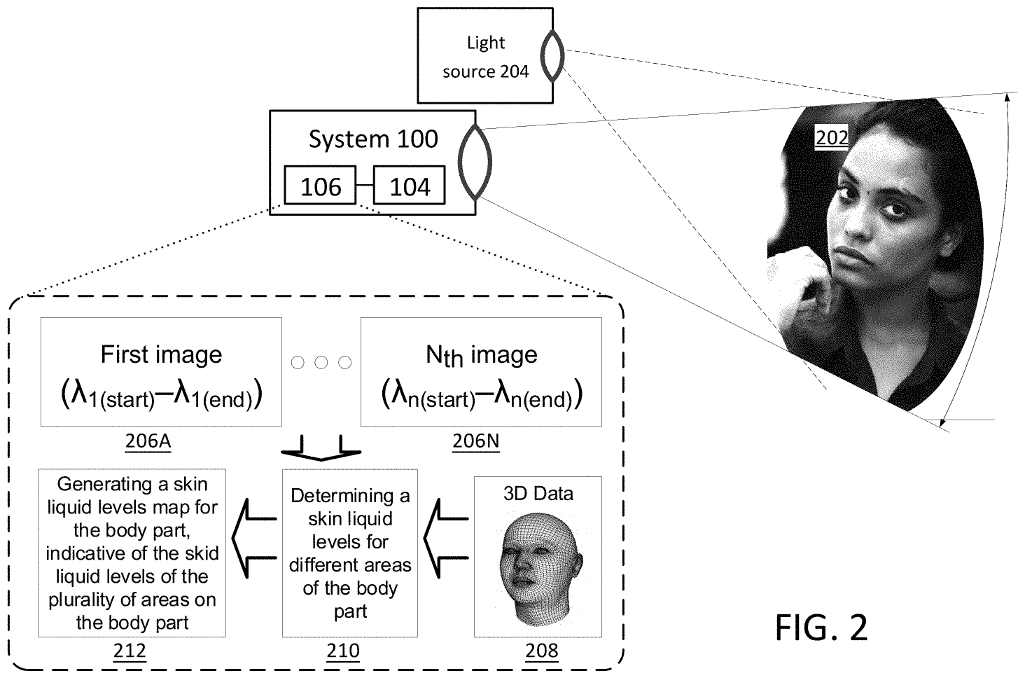

FIG. 2 illustrates system 100 and its operation when determining skin liquid levels (e.g., hydration, sebum) in a body part (in this case, a face 202), in accordance with examples of the presently disclosed subject matter. In the illustrated example, light is provided by an external light source 204 (e.g., LED, laser), but this is not necessarily so. A plurality of spectrally distinct images 206(denoted 206A through 206N) of body part 202 are captured (e.g., using one or more cameras; the illustration shows a non-limiting example of one camera). The spectral range of each image is denoted between λ(start) and λ(end). Nevertheless, as mentioned above, compound spectral ranges may optionally be implemented.

The images 206 may be taken from a single position/angle, or from a plurality of positions/angles. Optionally, the spectrally distinct images may be captured concurrently (e.g., using spectrally distinct optical filters). Optionally, the spectrally distinct images may be taken at different times (e.g., while illuminating the body part with spectrally distinct light sources, such as lasers or light emitting diodes—LEDs). The methods and systems disclosed below are not restricted to the example configuration illustrated in FIGS. 1 and 2, but rather FIGS. 1 and 2 serve as examples for systems in which the methods below may be implemented.

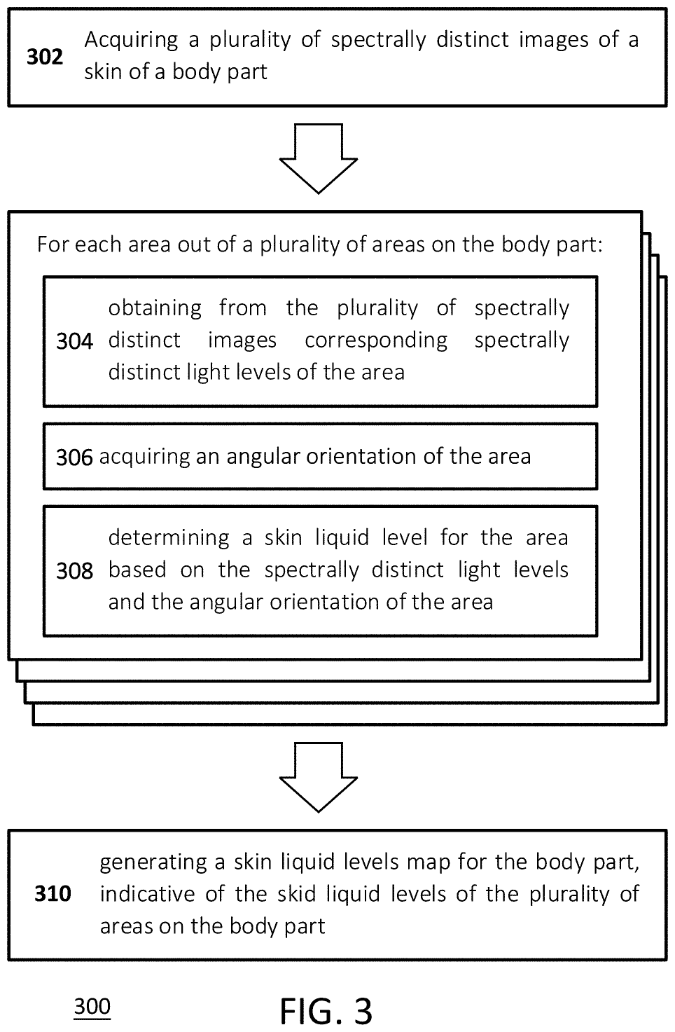

FIG. 3 illustrates method 300 for estimating skin liquid levels, in accordance with examples of the presently disclosed subject matter. Referring to the examples of the accompanying drawings, method 300 may optionally be carried out by system 100.

Stage 302 of method 300 includes acquiring a plurality of spectrally distinct images of a skin of a body part. The images may be acquired concurrently, but this is not necessarily so. While not necessarily so, the images may be detected by a sensor concurrently or within a relatively short time (under a second or under a minute). The images may be acquired from single position in space, but not necessarily so. The images may be acquired from the same angle, but this is not necessarily so. A plurality of areas of the body part are represented in multiple images out of the spectrally distinct images. Optionally, all of the areas of the body part for which the following stages are executed may be represented in all of the images, but this is not necessarily so. Areas of different sizes may be implemented. For example, the areas may be about 1 square centimeter (cm²), about 5 cm², about 10 cm², about 20 cm², and so on. For example, the areas may be about 1 pixel, about 5 pixels, about 10 pixels, about 100 pixels, and so on. Optionally, the areas of the body part for which the following stages are implemented may be of substantially the same size. However, this is not necessarily so. Referring to the examples of the accompanying drawings, stage 302 may optionally be carried out by one or more sensors 104 and/or by one or more external sensor (e.g., by data received via communication module 114). Referring to the examples of the accompanying drawings, the images may be images 206.

Following stage 302, stages 304, 306 and 308 are implemented for each area out of a plurality of areas on the body part.

Stage 304 includes obtaining from the plurality of spectrally distinct images corresponding spectrally distinct light levels of the area. Stage 304 may include obtaining spectrally distinct light levels (i.e., light levels from the respective images in which light of the relevant part of the spectrum is detected) from all of the spectrally distinct images in which the respective area is represented—or from some of them. Optionally, stage 304 may include obtaining light levels corresponding to the area from all of the spectrally distinct images, but this is not necessarily so. Stage 304 may include obtaining a single light level from each spectrally distinct image (e.g., a single measurement, a representative measurement out of few measurement, an average of multiple measurements such as multiple pixels), but a plurality of light levels may also be obtained from a single images (e.g., multiple pixel value of some or all of the pixels of the area). Referring to the examples of the accompanying drawings, stage 304 may optionally be carried out by processor 102.

Stage 306 includes acquiring an angular orientation of the area. The angular orientation may be representative of the orientation of the area with respect to the location of the sensor (e.g., with respect to an optical axis of the detection system), with respect to the image plane (e.g., if the latter is not perpendicular to the optical axis), with respect to the illumination axis, or to any other geometrical axis or plane in the system. It is noted that more than one angular orientation may be determined for a single area (e.g., one with respect to the optical axis, and on with respect to the direction of illumination, if the two are not parallel). It is noted that other three-dimensional (3D) data may also be obtained with respect to the area (e.g., distance from the sensor, distance from the source of illumination, and so on). Some ways in which the angular orientation of the area may be determined are discussed below. If the area is not flat, an average of its angular orientation may be determined, or any other representative angular orientation may be determined. Optionally, multiple angular orientations may be determined in order to represent the orientation of a non-flat area. Referring to the examples of the accompanying drawings, stage 306 may optionally be carried out by processor 102 or by an external system (e.g., by data received via communication module 114). Referring to the examples of the accompanying drawings, the orientation data as well as additional 3D parameters such as distance may be stored as 3D data 208.

Stage 308 includes determining a skin liquid level for the area, based on the spectrally distinct light levels and the at least one angular orientation of the area (and potentially also based on other 3D data of the area, e.g., distance from the detector, distance from the source of illumination). The angular orientation may allow, for example, to compensate for reduced light levels from body part areas which are oriented farther from being perpendicular to the optical axis and/or to the direction of illumination. For example, a computer algorithm (or a dedicated electric circuitry) may implement a skin liquid level assessing algorithm which receives as input light levels in the different distinct spectral ranges of the different spectrally distinct images. A preprocessing algorithm may be used to adjust the different light levels for compensating for effects of being inclined with respect to the illumination and/or with respect to the detecting sensor (which captures the respective image). Alternatively, an adjustment of the illumination levels based on the at least one angular orientation may be executed as part of the skin liquid level assessing algorithm. It is noted that the determining of the skin liquid level for a first area may be further based on other parts of one or more of the spectrally distinct images (e.g., corresponding to one or more body part areas). It is noted that adjusting for the angular orientation may take into account different types of reflections (or combinations thereof), such as specular reflection, Lambertian reflection, and so on. Referring to the examples of the accompanying drawings, stage 308 may optionally be carried out by processor 102. Referring to the examples of the accompanying drawings, stage 308 may correspond to the determining process 210 of FIG. 2.

For example, if the amount of reflected light at wavelength λ for an area having at least one angular orientation θ_{AREA,i} is reduced by a factor of estimated to be R(θ_{AREA,I,}λ), method 300 may include correcting the at least one light levels for the area for each of the spectrally distinct ranges by a factor of 1/R(θ_{AREA,I,}λ) (e.g., if narrow spectral ranges are used around wavelength λ. Implementation for wider spectral ranges can be implemented, mutatis mutandis). Any other suitable form of adjustments of the entire image or of individual areas based on the respective angular orientations may also be implemented.

After stages 304, 306 and 308 are executed for a plurality of areas of the body part, method 300 continues with stage 310. Stage 310 includes generating a skin liquid levels map for the body part, indicative of the skid liquid levels of the plurality of areas on the body part. Stage 310 may include generating skin liquid level maps (e.g., skin hydration maps, skin sebum level maps) of different types, such as an image, a table, a database entry, a graph, a histogram, and so on. It is noted that stage 310 may include generating a plurality of skin liquid level maps (e.g., for different types of fluid, in different depths within the skin, and so forth). Referring to the examples of the accompanying drawings, stage 310 may optionally be carried out by processor 102. Referring to the examples of the accompanying drawings, stage 310 may correspond to the generating process 212 of FIG. 2.

Method 300 may optionally continue with one or more of the following stages: displaying the skin liquid levels map, saving the skin liquid levels map to tangible memory storage, processing the skin liquid levels map (e.g., for determining a medical condition, for recommending a treatment, for matching a commercial product, for adjusting operational parameters of another system, for calibrating another system), and so on.

Optionally, the skin liquid levels of the plurality of areas are indicative of hydration levels of the plurality of areas. Optionally, the skin liquid levels of the plurality of areas are indicative of sebum levels of the plurality of areas.

Optionally, for the same spectrally distinct light levels are obtained for a first area and a for a second area of the body part, wherein different angular orientations are acquired for the first area and for the second area, and wherein the method including determining a first skin liquid level for the first area and a second skin liquid level for the second area, the first skin liquid level and the second skin liquid level differing by at least 5%.

Optionally, method 300 may further include illuminating the body part with spectrally distinct light beams (e.g., corresponding to the spectrally distinct spectral ranges of the spectrally distinct images), and capturing the spectrally distinct images corresponding to the spectrally distinct light beams. Spectral filter may optionally be used in addition to the different light beams, but this is not necessarily so. The spectrally distinct light beams may be issued by a plurality of corresponding light sources (e.g., lasers, LEDs).

Optionally, method 300 may further include capturing the spectrally distinct images using a detector array having a plurality of spectrally distinct filters. The filters may filter light before it reaches the entire detector array, before reaching individual photosites of the detector array (e.g., spectral array filter), or in any other suitable manner

Optionally, at least one of the spectrally distinct images depicts a reflection target, wherein the generating of the skin liquid levels is further based on reflection levels of the reflection targets in the at least one of the spectrally distinct images. For example, the reflection target may be white, reflecting over 95% (or 99%, etc.) of the light in all of the different distinct spectral ranges. Otherwise, the reflection rates of the reflection target may be known in advance. Angular orientation of the reflection target, if necessary, may be known in advance or determined as part of method 300. For example, the reflection levels from the reflection target may be used to calibrate light reflection levels from one or more areas of the body part.

Optionally, the method is a computer-implemented method for estimating skin liquid levels, including executing on a processor the steps of the method.

Reverting to stage 306, it is noted that the one or more angular orientations for each area may be acquired in different ways. Optionally, the angular orientations may be determined after the acquisition of the spectrally distinct images, but this is not necessarily so.

Optionally, the angular orientations may change between different times the body part is examined for skin liquid levels. For example, a user may scan her face (or other body part, such as arm, neck, or belly) for skin liquid levels using a portable camera-equipped system such as a smart phone, a laptop or a webcam). Every time the user uses the portable device, the 3D relative position between the portable device and the body part may change—both the distance and the angular orientation—which method 300 compensates for.

One example for determining angular orientation of the different areas includes acquiring the angular orientations of the areas by processing depth data captured by a detector (e.g., camera, other detector array) which captured at least one of the spectrally distinct images. The depth data may be captured by a time-of-flight sensor, by gated imaging, by processing reflections of a patterned illumination, or in any other suitable manner Knowing the distance to different locations of the body part enables determination of angular orientations of different surfaces of the body part. In another example, another type of depth sensor may be used (e.g., a lidar, a range finder). The other detector may be integrated into the same system as the detector array which captures the images (e.g., both being implemented in the same smartphone), but this is not necessarily so. The angular orientation may also be determined directly (and not by geometrical computation of multiple points in a 3D space). For example, direct determination of angular orientation may be implemented by processing temporal distortion of a reflected pulse of light.

In another example, the acquiring of the angular orientations for the plurality of areas may include applying a face-recognition algorithm to the body part, and assigning different angular orientations to different areas based on the results of the face recognition algorithm. Other type of body-part recognition algorithms may be used for other types of body parts (e.g., hand). For example, a face-recognition algorithm may be used to identify facial parts such as nose, mouth and eyes, and the angular orientations of those and other facial parts (e.g., forehead, cheeks, chin) may be assessed based on the result of the face recognition and on possibly based on additional image processing (e.g., light levels of regular visible light image).

In another example, the acquiring of the angular orientations for the plurality of areas may include retrieving previously sampled three dimensional (3D) information of the body part from a memory storage, and mapping the 3D information to at least one of the spectrally distinct images. For example, the user may be sampled once using her smartphone or in a service station to determine the 3D structure of her face (or other relevant body parts), and this 3D structure may be matched to the collected data (e.g., one or more of the spectrally distinct image, another visible light image).

A non-transitory computer-readable medium for estimating skin liquid levels is disclosed, including instructions stored thereon, that when executed on a processor, perform any combination discussed above of steps of method 300.

A program storage device readable by machine is disclosed, tangibly embodying a program of instructions executable by the machine to perform method for estimating skin liquid levels, comprising any combination discussed above of steps of method 300.

Reverting to FIG. 1, it is noted that system 100 includes at least processor 102 which is operable to and configured to:
l. Acquire a plurality of spectrally distinct images of a skin of a body part;
m. For each area out of a plurality of areas on the body part:
   i. obtain from the plurality of spectrally distinct images corresponding spectrally distinct light levels of the area,
   ii. acquire an angular orientation of the area, and
   iii. based on the spectrally distinct light levels and the angular orientation of the area, determine a skin liquid level for the area;
n. Generate a skin liquid levels map for the body part, indicative of the skid liquid levels of the plurality of areas on the body part.

While not necessarily so, system 100 may implement method 300. Different implementations of system 100 may implement any one or more of the variations of method 300 discussed above. In addition to the processor, system 100 may include any combination of the one or more components illustrated in FIG. 3, as well as additional components (e.g., a speaker for issuing instructions for a user, a battery). System 100 may be a portable communications device or a portable computer (e.g., a laptop, a smartphone, a tablet computer), but this is not necessarily so. Optionally, system 100 may include one or more detectors sensitive to infrared light, such as those developed by TriEye Ltd. of Tel Aviv, Israel. Optionally, the detectors (or any other component illustrated in FIG. 3) may be external to system 100 (e.g., an external camera).

Optionally, the skin liquid levels of the plurality of areas are indicative of hydration levels of the plurality of areas. Optionally, the skin liquid levels of the plurality of areas are indicative of sebum levels of the plurality of areas.

Optionally, system 100 may include a 3D processing module operable to process depth data captured by a detector which captured at least one of the spectrally distinct images, for determining angular orientations of different body part areas.

Optionally, the processor is operable to execute a face-recognition algorithm to the body part, and to assigning different angular orientations to different areas based on the results of the face recognition algorithm.

Optionally, system 100 may include a memory storage for storing previously sampled 3D information of the body, wherein the processor is configured to map the 3D information to at least one of the spectrally distinct images.

Optionally, for the same spectrally distinct light levels are obtained for a first area and a for a second area of the body part, wherein different angular orientations are acquired for the first area and for the second area, and wherein the processor determines a first skin liquid level for the first area and a second skin liquid level for the second area, the first skin liquid level and the second skin liquid level differing by at least 5%.

Optionally, system 100 may further include at least one detector for capturing the spectrally distinct images corresponding to the spectrally distinct light beams.

Optionally, system 100 may further include a plurality of spectrally distinct filters coupled to the at least one detector. System 100 may also include other optical components such as polarizers, lens, mirrors, prisms, and so on, for manipulating light before it is captured to one or more of the spectrally distinct images.

Optionally, system 100 may further include at least one light source for illuminating the body part with spectrally distinct light beams, and at least one detector for capturing the spectrally distinct images corresponding to the spectrally distinct light beams.

Optionally, at least one of the spectrally distinct images depicts a reflection target, wherein the processor is configured to generate the skin liquid levels further based on reflection levels of the reflection target in the at least one of the spectrally distinct images.

Optionally, at least two of the spectrally distinct images are indicative of detected light in infrared parts of the spectrum between 1000-1500 nm.

Optionally, the system is a portable communication device which includes the processor and at least one detector for capturing the spectrally distinct images corresponding to the spectrally distinct light beams.

While certain features of the disclosure have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure. It will be appreciated that the embodiments described above are cited by way of example, and various features thereof and combinations of these features can be varied and modified.

What is claimed is:

1. A method for estimating skin liquid levels, comprising:
acquiring a plurality of spectrally distinct images of a skin of a body part; and,
for each area out of a plurality of areas on the body part,
obtaining, from the plurality of spectrally distinct images, corresponding spectrally distinct light levels of the area;
acquiring an angular orientation of the area;
based on the spectrally distinct light levels and the angular orientation of the area, determining a skin liquid level for the area; and
based on the skin liquid level for each area, generating a skin liquid levels map for the body part indicative of the skid liquid levels of the plurality of areas on the body part.

2. The method of claim 1, wherein the skin liquid levels of the plurality of areas are indicative of hydration levels of the plurality of areas.

3. The method of claim 1, wherein the skin liquid levels of the plurality of areas are indicative of sebum levels of the plurality of areas.

4. The method of claim 1, wherein the acquiring of the angular orientation of the area comprises processing depth data captured by a detector that captured at least one of the spectrally distinct images.

5. The method of claim 1, wherein the acquiring of the angular orientations for the plurality of areas comprises applying a face-recognition algorithm to the body part, and assigning different angular orientations to different areas based on the results of the face recognition algorithm.

6. The method of claim 1, wherein the acquiring of the angular orientations for the plurality of areas comprises retrieving previously sampled three-dimensional (3D) information of the body part from a memory storage, and mapping the 3D information to at least one of the spectrally distinct images.

7. The method of claim 1, wherein for the same spectrally distinct light levels are obtained for a first area and a for a second area of the body part, wherein different angular orientations are acquired for the first area and for the second area, and wherein the method comprising determining a first skin liquid level for the first area and a second skin liquid level for the second area, the first skin liquid level and the second skin liquid level differing by at least 5%.

8. The method of claim 1, further comprising illuminating the body part with spectrally distinct light beams, and capturing the spectrally distinct images corresponding to the spectrally distinct light beams.

9. The method of claim 1, further comprising capturing the spectrally distinct images using a detector array having a plurality of spectrally distinct filters.

10. The method of claim 1, wherein at least one of the spectrally distinct images depicts a reflection target, wherein the generating of the skin liquid levels is further based on reflection levels of the reflection targets in the at least one of the spectrally distinct images.

11. The method of claim 1, wherein the method is a computer-implemented method for estimating skin liquid levels, comprising executing on a processor the steps of the method.

12. The method of claim 1, wherein at least two of the spectrally distinct images are indicative of detected light in infrared parts of the spectrum between 1000-1500 nm.

13. A system for estimating skin liquid levels, comprising:
a processor operable to perform a method that includes:
acquiring a plurality of spectrally distinct images of a skin of a body part; and, for each area out of a plurality of areas on the body part,
obtaining, from the plurality of spectrally distinct images, corresponding spectrally distinct light levels of the area;
acquiring an angular orientation of the area;
based on the spectrally distinct light levels and the angular orientation of the area, determining a skin liquid level for the area; and
based on the skin liquid level for each area, generating a skin liquid levels map for the body part indicative of the skid liquid levels of the plurality of areas on the body part.

14. The system of claim 13, wherein the skin liquid levels of the plurality of areas are indicative of hydration levels of the plurality of areas.

15. The system of claim 13, wherein the skin liquid levels of the plurality of areas are indicative of sebum levels of the plurality of areas.

16. The system of claim 13, comprising a 3D processing module operable to process depth data captured by a detector which captured at least one of the spectrally distinct images, for determining angular orientations of different body part areas.

17. The system of claim 13, wherein the processor is operable to execute a face-recognition algorithm to the body part, and to assigning different angular orientations to different areas based on the results of the face recognition algorithm.

18. The system of claim 13, wherein for the same spectrally distinct light levels are obtained for a first area and a for a second area of the body part, wherein different angular orientations are acquired for the first area and for the second area, and wherein the processor determines a first skin liquid level for the first area and a second skin liquid level for the second area, the first skin liquid level and the second skin liquid level differing by at least 5%.

19. The system of claim 13, further comprising at least one detector for capturing the spectrally distinct images corresponding to the spectrally distinct light beams.

20. The system of claim 13, further comprising at least one light source for illuminating the body part with spectrally distinct light beams, and at least one detector for capturing the spectrally distinct images corresponding to the spectrally distinct light beams.

21. The system of claim 13, wherein at least one of the spectrally distinct images depicts a reflection target, wherein the processor is configured to generate the skin liquid levels further based on reflection levels of the reflection target in the at least one of the spectrally distinct images.

22. The system of claim 13, wherein at least two of the spectrally distinct images are indicative of detected light in infrared parts of the spectrum between 1000-1500 nm.

23. The system of claim 13, wherein the system is a portable communication device which comprises the processor and at least one detector for capturing the spectrally distinct images corresponding to the spectrally distinct light beams.

24. A program storage device readable by machine, tangibly embodying a program of instructions executable by the machine to perform a method for estimating skin liquid levels, comprising:
acquiring a plurality of spectrally distinct images of a skin of a body part; and,
for each area out of a plurality of areas on the body part,
obtaining, from the plurality of spectrally distinct images, corresponding spectrally distinct light levels of the area;
acquiring an angular orientation of the area;
based on the spectrally distinct light levels and the angular orientation of the area, determining a skin liquid level for the area; and
based on the skin liquid level for each area, generating a skin liquid levels map for the body part indicative of the skid liquid levels of the plurality of areas on the body part.
